(19) United States
(12) Patent Application Publication
(10) Pub. No.: US 2026/0263753 A1
(43) Pub. Date: Sep. 10, 2026

(54) RETRACTABLE NEEDLE PROTECTOR ASSEMBLY

(71) Applicant: POLY MEDICURE LIMITED, Faridabad, Haryana (IN)

(72) Inventor: Rishi BAID, New Delhi (IN)

(73) Assignee: POLY MEDICURE LIMITED, Faridabad, Haryana (IN)

(21) Appl. No.: 19/161,091

(22) PCT Filed: Feb. 29, 2024
(86) PCT No.: PCT/IB2024/051944

§ 371 Date: Aug. 29, 2025

(30) Foreign Application Priority Data
Mar. 1, 2023 (IN) 202311013879

(51) Int. Cl. A61M 25/06

(52) U.S. Cl. CPC A61M 25/0631; A61M 25/0606; A61M 25/0637; A61M 2205/273; A61M 2205/8281

(57) ABSTRACT

A retractable needle protector assembly for use with a blood collection set is provided that includes a wing housing, a protective housing, a needle having a proximal end connected to a needle hub and a distal end defining a tip wherein the needle hub is movable with the needle relative to the wing housing and the protective housing between a position of use and/or before use position in which at least the tip of the needle is outside of the wing housing, and an after use and/or retracted position in which the needle with needle tip is fully received in the wing housing and the protective housing upon activation of a push button.

### FIELD OF THE INVENTION

The present invention relates to safety needle devices for safe and convenient handling of needles. More particularly, the present invention relates to a retractable needle protector assembly used in blood collection and medicinal injection procedures comprising a needle, for example of a disposable medical device, a wing housing and a protective housing, wherein the needle having a tip at its distal end is attached to a needle hub being movable relative to the wing housing and protective housing between a position of use in which at least the tip of the needle is outside of the wing housing, and a retracted position in which the needle is fully received in the wing housing and protective housing by use of a push button engageably connected to the needle hub and the wing housing. The needle protector assembly is used in a blood collection set for the collection of blood drawn from a patient or can be used for infusion of fluid including medicinal fluids, medicines, blood etc.

### BACKGROUND OF THE INVENTION

In today's hospitals and medical set-ups, a wide variety of disposable needle and syringe devices are routinely used to administer medication by injection, for intravenous blood collection, and for insertion of catheter devices.

The increasing incidence of blood borne pathogens such as Human Immunodeficiency Virus (HIV), combined with Hepatitis B Virus (HBV) and Hepatitis C Virus (HCV), presents healthcare workers with an occupational hazard unprecedented in modern medicine. An accidental contact with a used needle can cause the transmission of a disease such as hepatitis, HIV, etc.

There are some other known blood borne pathogens which are transmitted via blood and bodily fluids. The presence of any of these pathogens in patients poses a risk to healthcare workers when invasive procedures are performed. Infectious diseases are now one of the leading cause of death, behind heart disease and cancer, signifying a growing need for safer medical devices.

With the widespread use of disposable hypodermic needles and syringes, there exists a constant need for ways to safely and conveniently dispose of such sharp implements after use without risk of exposing medical personal handling them to injury, infection or disease by puncture or contact with a used needle or syringe. Today's health professionals, especially those who attend to patients suffering from highly contagious diseases such as Acquired Immune Deficiency Syndrome (“AIDS”) and Hepatitis, are acutely aware of such risks.

Once a needle and/or syringe is used, both implements are contaminated and must be disposed of in a safe manner. It's been a common practice to break or cut the needle after use before transport to ultimate disposal in order to eliminate the sharp end point and thereby reduce the risk of puncture, scratching or other injury which might result from handling such sharp end point devices. Such practices are now disfavored because of the substantial danger of an accidental puncture during the breaking or cutting operations, and the recognition that the cutting tool itself can be a breeding ground for disease-causing micro-organisms.

As the population of infected individuals increases, more people will be treated by healthcare workers, which imposes further risks of disease transmission from patients to healthcare workers. Also, the use of disposable medical devices is increasing every year. Additionally, a remarkable number of patient to patient transmissions in the healthcare setting have been identified throughout the world. Early data suggests improper infection control techniques contribute directly to this increase: including improper use of medical devices having needles, multiple-dose medicine vials, and failure to change protective gloves and gear for each new patient.

Healthcare workers are increasingly at risk of disease transmission, and nurses perform the majority of invasive hypodermic procedures, such as injecting medicine, collecting blood and inserting indwelling intravenous (I.V.) catheters.

Nurses and other healthcare personnel are routinely injured by the exposed, sharp lancet of the needle after use on a patient. The critical time where a percutaneous injury can occur is from the moment the needle is withdrawn from the patient, or I.V. port, to the time the contaminated needle is safely discarded.

Prior to the proliferation of HIV and serum hepatitis, a needle stick injury was considered a routine part of providing patient care. A needle stick injury now carries a life-threatening consequence and healthcare workers must live with this terror on a daily basis.

In view of the above, a need exists for a needle protector assembly which helps to prevent accidental contact of a medical professional or any other person with the sharp tip of the needle after use of the disposable medical device. There is a requirement for a blood collection set including a retractable needle protector assembly that provides secure and effective protection for a used needle cannula, which is easy and cost-effective to manufacture and simple and safe to operate.

### OBJECTS AND SUMMARY OF THE INVENTION

It is an object of the invention to provide a retractable needle protector assembly which provides reliable protection against accidental pricking by a needle, for example of a disposable medical device, and which is easy and inexpensive to manufacture and simple and safe to operate.

A retractable needle protector assembly for use with a blood collection set comprising a wing housing, a protective housing, a needle having a proximal end connected to a needle hub and a distal end defining a tip wherein the needle hub is movable with the needle relative to the wing housing and the protective housing between a position of use and/or before use position in which at least the tip of the needle is outside of the wing housing, and an after use and/or retracted position in which the needle with needle tip is fully received in the wing housing and the protective housing upon activation of a push button. The push button is lockingly received within the wing housing through an opening thereof and engageably connected to the needle hub and the wing housing. Thus, the push button engageably attached to the wing housing through the opening being configured to movably receive the needle hub in a locking engagement in the before use position.

The push button is lockingly received within the wing housing through an opening thereof and engageably connected to the needle hub and the wing housing. The wing housing having a tubular main body and two wings extending in opposite radial directions R from the main body.

The wing housing has a proximal opening and a distal opening to movably house the needle hub allowing the needle having the needle tip to pass through the distal opening thereof. The protective housing having a tubular body has a proximal opening and a distal opening movably house the needle hub with the needle in an after use and/or retracted position. A spring is movably arranged within the wing housing over the needle hub in a distal region thereof.

The push button has a substantially rectangular outer profile having a front and a back portion, wherein a push interface is provided in the front portion having a semi-circular profile and a locking member provided opposite to the push interface in the back portion and an opening is provided in between the push interface and the locking member.

The opening in the push button made of a profile having a smaller diameter opening and a bigger diameter opening, wherein both the smaller diameter opening and bigger diameter opening adjoins in an area between the push interface and the locking member, wherein the smaller diameter opening part is adjacent the locking member and the bigger diameter opening part is adjacent the push interface.

A blood collection set comprising a retractable needle protector assembly, a flexible tube extending from and being connected to a proximal end of the protector assembly, a connection assembly mounted on the other end of the tube and a protective cover removably mounted to the needle portions of the protector assembly opposite tube, such as through frictional engagement.

### BRIEF DESCRIPTION OF THE DRAWINGS

Embodiments of the present disclosure now will be described more fully hereinafter with reference to the accompanying drawings, in which embodiments of the present disclosure are shown. This present disclosure may, however, be embodied in many different forms and should not be construed as limited to the embodiments set forth herein. Rather, these embodiments are provided so that this disclosure will be thorough and complete, and will fully convey the scope of the claims to those skilled in the art. Like numbers refer to like elements throughout. The embodiments of the invention are described in the following description and in the accompanying drawings, wherein:

FIGS. 1A & 1B illustrate a front and side view respectively of a blood collection set having a retractable needle protector assembly according to the present invention.

FIG. 2 illustrates a cross-sectional front view of the blood collection set having a retractable needle protector assembly of FIG. 1A according to the present invention.

FIGS. 3A & 3B illustrate a top and perspective view respectively of a push button used in the retractable needle protector assembly according to the present invention.

FIG. 4A illustrates a front view of the retractable needle protector assembly in a before use position and in particularly before activation of the push button according to the present invention.

FIG. 4B illustrates a cross-sectional partial side view of the retractable needle protector assembly in a before use position and in particularly before activation of the push button according to the present invention.

FIG. 5A illustrates a front view of the retractable needle protector assembly in an after-use position and in particularly after activation of the push button according to the present invention.

FIG. 5B illustrates a cross-sectional partial side view of the retractable needle protector assembly in an after-use position and in particularly after activation of the push button according to the present invention.

FIG. 5C illustrates a partial view of the retractable needle protector assembly in an after-use position according to the present invention.

FIG. 6 illustrates an exploded perspective view of the retractable needle protector assembly according to the present invention.

### DETAILED DESCRIPTION OF THE INVENTION

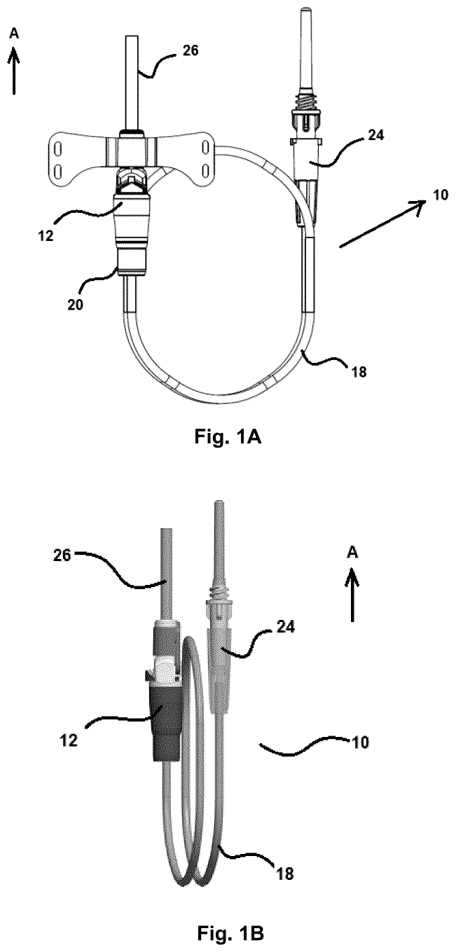

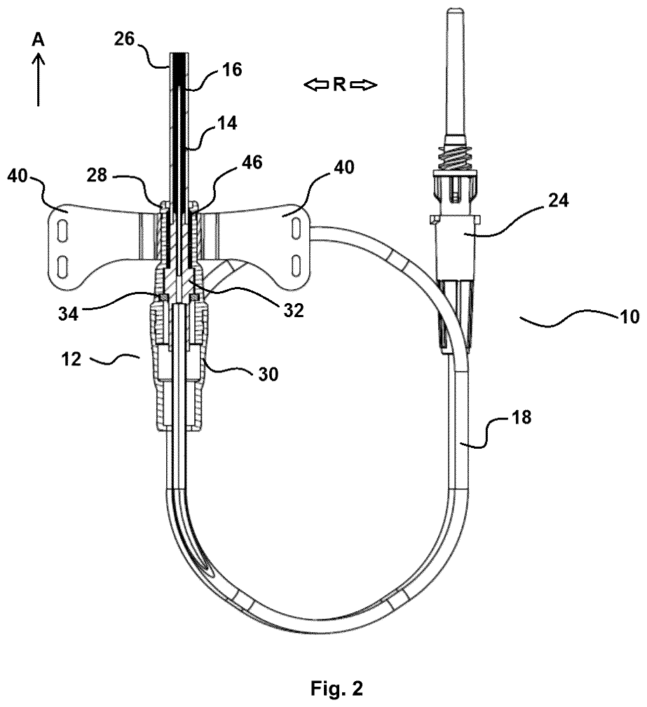

In FIGS. 1A and 1B front and side views respectively of a blood collection set 10 comprising a retractable needle protector assembly 12 according to the present invention is shown. In FIG. 2 a cross-sectional view of the blood collection set 10 having the retractable needle protector assembly 12 according to the present invention is shown. As can be seen, the retractable needle protector assembly 12 is part of a disposable medical device, for example a blood collection set 10, having a hypodermic needle 14 (shown in FIG. 2) and is provided to prevent accidental contact of a medical professional or any other person with the sharp tip 16 of the needle 14 after use of the disposable medical device i.e. blood collection set 10.

In this context, the end of the needle 14 having the sharp tip 16 of the needle is denoted as the distal end, whereas the opposite end of the needle 14 is denoted the proximal end. Likewise, the orientation of parts of other structural elements facing towards the needle tip 16 will be referred to as distal, whereas the orientation of parts of these structural elements facing in the opposite direction will be referred to as proximal.

The needle 14 extends in an axial direction towards the tip 16, thereby defining an axial direction A for the needle protector assembly 10. A direction perpendicular to the axial direction A is denoted as radial R.

As shown in FIGS. 1A and 11B, a blood collection set 10 comprises a retractable needle protector assembly 12, a flexible tube 18 extending from a proximal end 20 of the protector assembly 12 and being connected to a proximal end of a needle hub 32, a connection assembly 24 mounted on the other end of the tube 18 and a protective cover 26 removably mounted to the needle portions of the protector assembly 12 opposite tube 18, such as through frictional engagement etc.

While described herein in terms of one of the embodiments of a blood collection set 10, the retractable needle protector assembly 12 of the present invention may be used with or incorporate other medical devices used in connection with a needle 14, such as a connection assembly 24 comprising a luer lock (male or female), hypodermic syringe assembly, a hypodermic needle, a single and/or double ended needle assembly for blood collection, an intravenous infusion set, needle less injection, or other handling devices or medical device assemblies that may or may not contain piercing elements.

As shown in FIG. 2, the retractable needle protector assembly 12 of blood collection set 10 includes a wing housing 28, a protective housing 30, a needle 14 having a proximal end connected to a needle hub 32 and a distal end defining a tip 16 wherein the needle hub 32 is movable with the needle 14 relative to the wing housing 28 and the protective housing 30 between a position of use and/or before use position in which at least the tip 16 of the needle 14 is outside of the wing housing 28, and an after use and/or retracted position in which the needle 14 with needle tip 16 is fully received in the wing housing 28 and the protective housing 30 u[pm activation of a push button 34. The push button 34 is lockingly received within the wing housing 28 through an opening 36 (as shown in FIG. 6) thereof and engageably connected to the needle hub 32 and the wing housing 28.

Thus, the push button 34 engageably attached to the wing housing 28 through the opening 36 being configured to movably receive the needle hub 32 in a locking engagement in the before use position.

In the region of its proximal end, the needle 14 is connected in a leak free manner, for example glued, to the needle hub 32. The needle 14 has a lumen extending through needle 14 from proximal to distal end. The proximal end of the needle 14 is beveled to define a sharp puncture tip 16, such as intravenous puncture tip. Puncture tip 16 is provided for insertion into a patient's skin and blood vessel, such as a vein, and is therefore designed to provide ease of insertion and minimal discomfort during venipuncture.

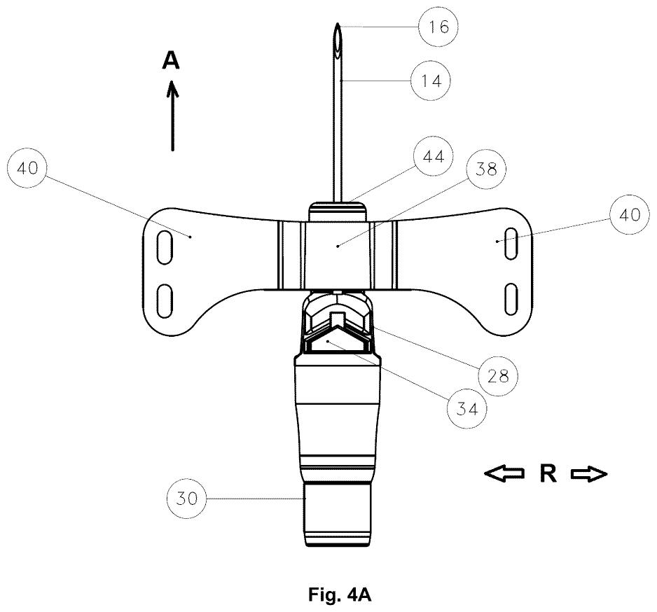

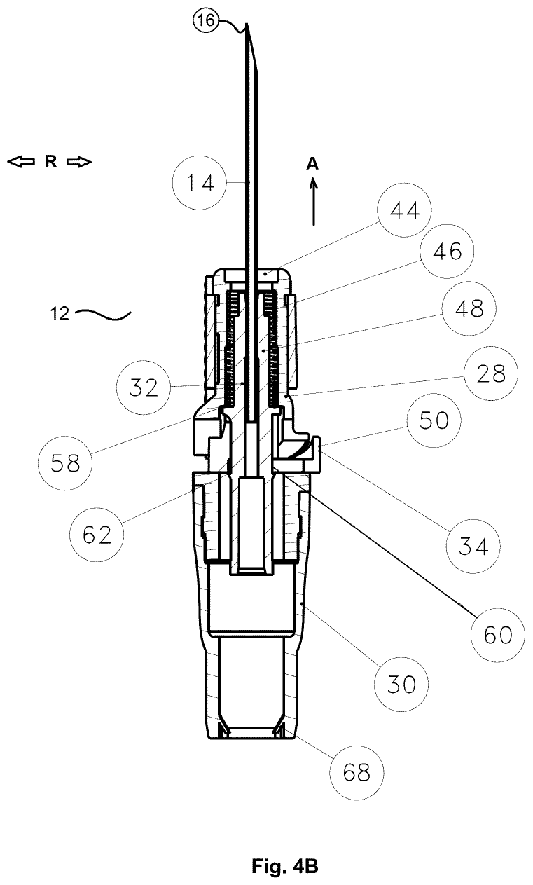

The wing housing 28 as shown in FIG. 4A of this embodiment, not being limited to, has a tubular main body 38 and two wings 40 which extend in opposite radial directions R from the main body 38. As shown in FIG. 6, the wing housing 28 has a proximal opening 42 and a distal opening 44 being configured to movably house the needle hub 32 allowing the needle 14 having the needle tip 16 to pass through the distal opening 44 thereof. The wings 40 can either be formed integrally with the wing housing 16, or they can be separate elements that are attached to the wing housing 40. In alternate embodiments, the wing housing 28 may include a catheter tube configured at a distal end thereof allowing the needle 14 to pass therethrough.

The protective housing 30 likewise as shown in FIG. 6 having, not being limited to, a tubular body has a proximal opening 42 and a distal opening 44 being configured to movably house the needle hub 32 with the needle 14 in an after use and/or retracted position. The after use and/or retracted position refers to the position/state when the needle 14 with its sharp tip 16 is retracted fully within the wing housing 28 and the protective housing 30. The proximal opening 42 of the wing housing 28 is engageably attached/connected with the distal opening 44 of the protective housing 30 in a leak proof manner by locking arrangements such as snap fit, threaded engagement etc.

The shape of wing housing 28 and protective housing 30 is not limited to having a tubular body and may include other shapes such as square, conical, rectangular etc. The shape and diameter of the tubular body may be varied along its length, and the cross-sectional area of the tubular body may be reduced in one or more selected areas as per dimensional requirements.

A spring 46 is movably arranged within the wing housing 28 over the needle hub 32 in a distal region 48 thereof. The spring 46 stores energy when in compressed state, such as when the retractable needle protector assembly 12 is in a before use position as shown in FIG. 2 with the needle 14 having the needle tip 16 exposed out from the distal opening 44 of the wing housing 28 for a venipuncture procedure.

Further as shown, one end of the flexible tube 18 is attached in a leak free manner, for example glued, to a proximal end portion of the needle hub 32. The other end of the tube 18 may be connected to a connection assembly 24 comprising, for example, a luer lock, a single and/or double ended needle assembly for blood collection or any other component to facilitate flow of fluid, such as medicine or blood, to or from a patient or donor.

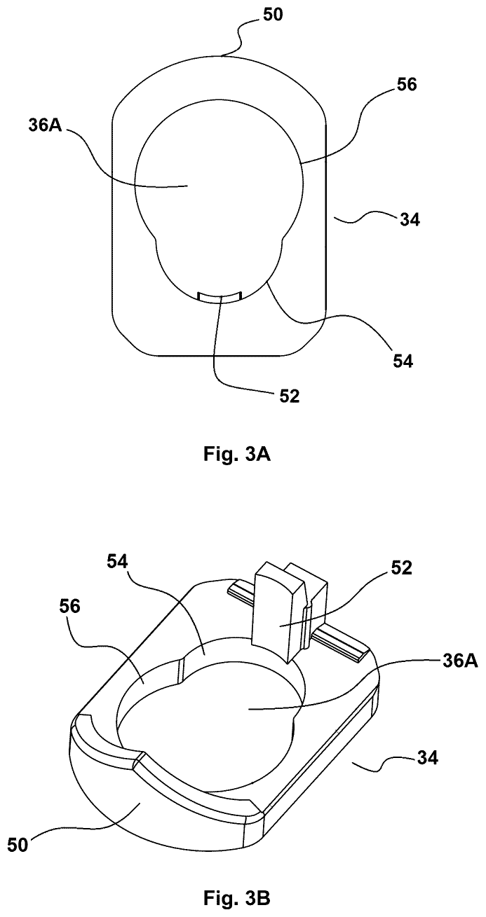

FIGS. 3A and 3B illustrate a top and perspective view respectively of a push button 34 used in the retractable needle protector assembly 12 according to the present invention. As can be seen, the push button 34, not being limited to, has a substantially rectangular outer profile having a front and a back portion, wherein a push interface 50 is provided in the front portion having a semi-circular profile and a locking member 52 provided opposite to the push interface 50 in the back portion and an opening 36A is provided in between the push interface 50 and the locking member 52. The opening 36A is made of a profile having a smaller diameter opening 54 and a bigger diameter opening 56. Both the smaller diameter opening 54 and bigger diameter opening 56 adjoins in an area between the push interface 50 and the locking member 52. The smaller diameter opening 54 part is adjacent the locking member 52 and the bigger diameter opening 56 part is adjacent the push interface 50. As can be seen, the profile and/or dimension of the smaller diameter opening 54 is smaller than the profile and/or dimension of the bigger diameter opening 56. The push interface 50 has a substantially semi-circular outer profile and is configured to be of use for pressing the push button 34 by a finger and/or thumb of a user using the device/assembly 12. The shape and dimension of the push button 34 is not limited to the shape, dimension and inner and/or outer profiles discussed herein and embodies to include other shapes, dimension and inner and/or outer profile for example, the push button 34 can have a circular, oval or any other shape outer profile.

Referring now to FIG. 4A which show a front view of the retractable needle protector assembly 12 in a before use position and in particularly before activation of the push button 34 according to the present invention and FIG. 4B which illustrates a cross-sectional partial side view of the retractable needle protector assembly 12 in a before use position and in particularly before activation of the push button 34 according to the present invention. As can be seen in FIG. 4A, retractable needle protector assembly 12 is in a before use position with the needle 14 having the needle tip 16 exposed out from the distal opening 44 of the wing housing 28 being ready for a venipuncture procedure.

Further, as can be seen in FIG. 4B, the wing housing 28 is engageably connected to the protective housing 30 in a leak proof manner having a needle hub 32 movably arranged therein. The needle hub 32, not being limited to, is provided with a retaining element 58, a locking element 60 and a blocking element 62 in between the proximal and distal end thereof. The needle hub 32 is lockingly engaged with the push button 34 through the locking element 60 and movably housed within the wing housing 28 and the protective housing 30. As can be seen, the needle hub 32 movably receives and retain a spring 46 in a compressed state in a distal region 48 thereof with the retaining element 58.

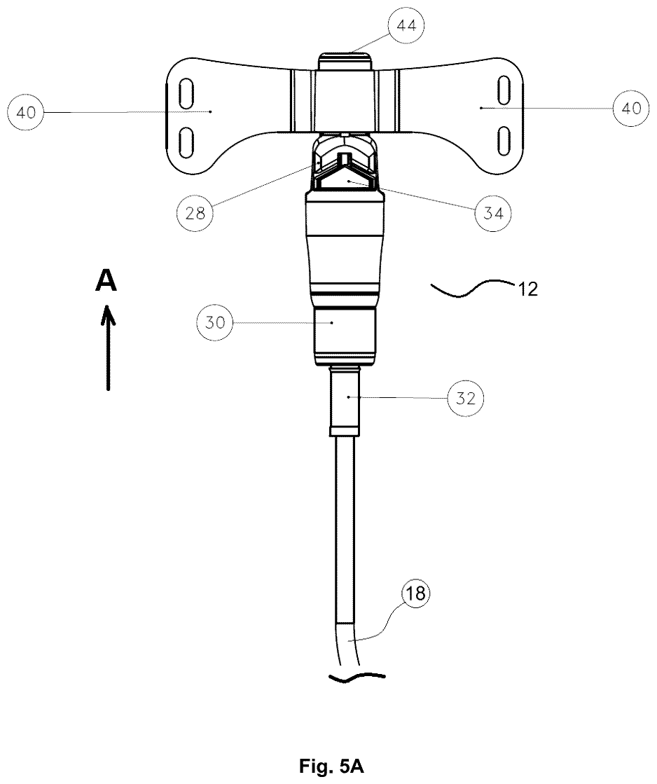

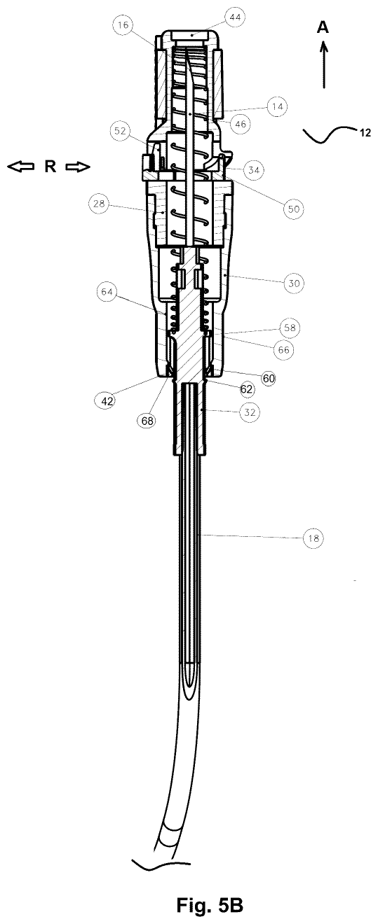

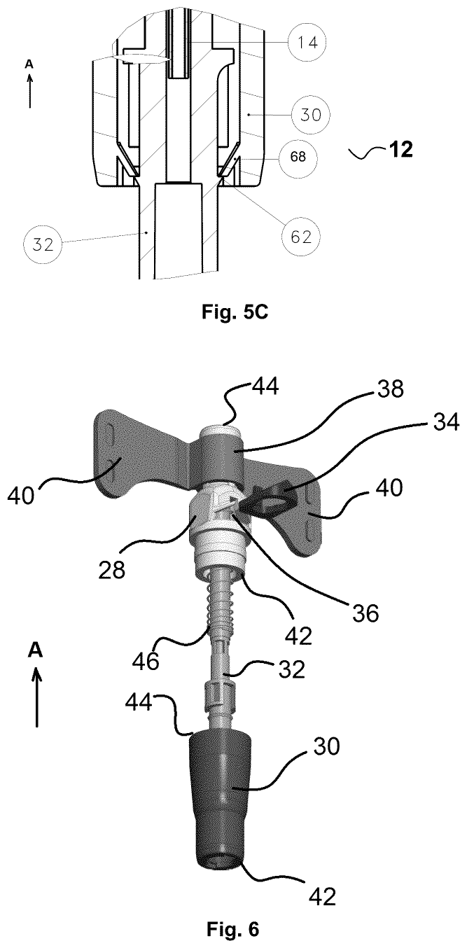

The spring 46 being in a compressed state is movably arranged between the wing housing 28 and the needle hub 32. The push button 34 has a push interface 50 which can be used by a user of the device/assembly 12 to activate the push button 34 by using his/her finger or thumb upon completion of the blood collection procedure. The stored energy of the compressed spring 46 is released upon radial movement R of the push button 34 once the push interface 50 is pressed/pushed, thereby forcing the needle hub 32 to retract with the needle 14 entirely within the wing housing 28 and protective housing 30 to an after-use position (as shown in FIGS. 5A & 5B) protectively covering the puncture tip 16 of the needle 14 therein. The retraction of the needle hub 32 with the needle 14 takes place in a direction opposite to the axial direction A.

Reference is now made to FIG. 5A, which illustrates a front view of the retractable needle protector assembly 12 in an after-use position and in particularly after activation of the push button 34 according to the present invention. Reference is also made to FIG. 5B, which illustrates a cross-sectional partial side view of the retractable needle protector assembly 12 in an after-use position and in particularly after activation of the push button 34 according to the present invention. As can be seen in FIG. 5A, retractable needle protector assembly 12 is in an after use position with the needle 14 having the needle tip 16 retracted within the wing housing 28 and protective housing 30 protectively covering the puncture tip 16 of the needle 14 therein once the push button 34 is activated/pressed/pushed.

Further, as can be seen from FIG. 5B, post a successful venipuncture and completion of the blood collection procedure, the push interface 50 of the push button 34 is pressed in a radial direction R by a user of the device/assembly 12 activating the push button 34. The radial movement of the push button 34 due to such pressing activates the push button 34 wherein the stored energy of the compressed spring 46 is released thereby forcing the needle hub 32 to retract with the needle 14 entirely within the wing housing 28 and the protective housing 30 to an after-use position protectively covering the puncture tip 16 of the needle 14 therein. As shown, the retraction of the needle hub 32 with the needle 14 takes place in a direction opposite to the axial direction A. The push button 34 is provided with a locking member 52 (as shown in FIGS. 3A & 3B) configured to lockingly engage with the wing housing 28.

As to explain the retraction of the needle 14 with the needle hub 32 in the wing housing 28 and protective housing 30, the push button 34 once pressed and activated due to the radial movement R which takes place due to press/push force exerted by a user of the device/assembly 12 which forces the locking element 60 of the needle hub 32 to disengage from the push button 34. Prior to activation of the push button 34, the needle hub 32 with the locking element 60 is retained lockingly by the smaller diameter opening 54 part of the push button 34 as shown in FIGS. 3A & 4A. Post activation of the push button 34 causes the movement of the push button 34 in the radial direction R, wherein lockingly retained needle hub 32 moves into the bigger diameter opening 56 part of the push button 34 disengaging the locking element 60 of the needle hub 32. Due to such disengagement of the locking element 60 of the needle hub 32 and shifting of the needle hub 32 into the bigger diameter opening 56 part of the push button 34 releases the stored energy of the compressed spring 46 which causes expansion of the compressed spring 46 creating a retracting force which forces the needle 14 with the needle hub 32 to retract entirely through the bigger diameter opening 56 of the push button 34 within/into the wing housing 28 and the protective housing 30 in an after-use position. As mentioned above, in the after-use position the needle hub 32 with the needle tip 16 is protectively secured within the wing housing 28 and protective housing 30. As can be seen, in the after-use retracted position, locking element 60 of the needle hub 32 with the proximal end part thereof is exposed out of the proximal opening 42 of the protective housing 30. The protective housing 30 is having an inner 64 and outer wall 66. The inner wall 64 of the protective housing 30 is provided with a blocking member 68, not being limited to, adjacent the proximal end of the protective housing 30 as shown in FIG. 4B. Once retracted post activation of the push button 34 and in particularly being in an after use position, the blocking member 68 of the protective housing 30 blocks the movement of the needle hub 32 in the axial direction A causing the needle hub 32 with the needle 14 having the tip 16 being retained protectively within the wing housing 28 and protective housing 30. In particularly, in the after use position, the movement of the blocking element 62 of the needle hub 32 in axial direction A is blocked by the blocking member 68 of the protective housing 30 as shown in FIG. 5C. It is to be understood that one or more blocking member can be provided anywhere in between the proximal and distal end of the inner wall of the protective housing 30. Likewise, needle hub 32 can be provided with one or more blocking element anywhere between the proximal and distal end thereof.

Being in such a state i.e. in the after use position, even under excessive force if exerted on the needle hub 32 in the axial direction A, the needle hub 32 with the needle 14 having the tip 16 continue being lockingly retained protectively within the wing housing 28 and protective housing 30 preventing completely the risk of accidental pricks by the sharp tip 16 of the needle 14.

Once activated, the push button 34 is locked with the wing housing 28 through the locking member 52 provided therein such that the push button 34 cannot be re-used and thus ensuring that the sharp tip 16 of the needle 14 is protectively protected and retained within the wing housing 28 and protective housing 30 removing completely any risk of accidental pricking by the sharp tip 16 of the needle 14.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of the present disclosure. As used herein, the term “and/or” includes any and all combinations of one or more of the associated' listed items.

The terminology used herein is for the purpose of describing particular embodiments only and is not intended to be limiting of the present disclosure. As used herein, the singular forms “a”, “an” and “the” are intended to include the plural forms as well, unless the context clearly indicates otherwise.

It will be further understood that the terms “comprises” “comprising,” “includes” and/or “including” when used herein, specify the presence of stated features, integers, steps, operations, elements, and/or components, but do not preclude the presence or addition of one or more other features, integers, steps, operations, elements, components, and/or groups thereof Unless otherwise defined, all terms (including technical and scientific terms) used herein have the same meaning as commonly understood by one of ordinary skill in the art to which this present disclosure belongs. It will be further understood that terms used herein should be interpreted as having a meaning that is consistent with their meaning in the context of this specification and the relevant art and will not be interpreted in an idealized or overly formal sense unless expressly so defined herein.

### REFERENCE NUMERALS

A axial direction
R radial direction/radial/radial movement
10 blood collection set
12 retractable needle protector assembly/protector assembly
14 needle
16 tip of needle/sharp tip/needle tip/puncture tip
18 flexible tube/tube
20 proximal end
22 distal end
24 connection assembly
26 protective cover
28 wing housing
30 protective housing
32 needle hub
34 push button
36 opening
36A opening
38 main body
40 wing(s)
42 proximal opening
44 distal opening
46 spring
48 distal region
50 push interface
52 locking member
54 smaller diameter opening
56 bigger diameter opening
58 retaining element
60 locking element
62 blocking element
64 inner wall
66 outer wall
68 blocking member

1. A retractable needle protector assembly for use with a blood collection set comprising a wing housing, a protective housing, a needle having a proximal end connected to a needle hub and a distal end defining a tip wherein the needle hub is movable with the needle relative to the wing housing and the protective housing between a position of use and/or before use position in which at least the tip of the needle is outside of the wing housing, and an after use and/or retracted position in which the needle with needle tip is fully received in the wing housing and the protective housing upon activation of a push button.

2. The retractable needle protector assembly as claimed in claim 1, wherein the push button is lockingly received within the wing housing through an opening thereof and engageably connected to the needle hub and the wing housing.

3. The retractable needle protector assembly as claimed in claim 1, wherein the wing housing having a tubular main body and two wings extending in opposite radial directions R from the main body.

4. The retractable needle protector assembly as claimed in claim 1, wherein the wing housing (28) has a proximal opening and a distal opening to movably house the needle hub allowing the needle having the needle tip to pass through the distal opening thereof.

5. The retractable needle protector assembly as claimed in claim 1, wherein the protective housing having a tubular body has a proximal opening and a distal opening to movably house the needle hub with the needle in an after use and/or retracted position.

6. The retractable needle protector assembly as claimed in claim 1, wherein a spring is movably arranged within the wing housing over the needle hub in a distal region thereof.

7. The retractable needle protector assembly as claimed in claim 1, wherein the push button has a substantially rectangular outer profile having a front and a back portion, wherein a push interface is provided in the front portion having a semi-circular profile and a locking member provided opposite to the push interface in the back portion and an opening is provided in between the push interface and the locking member.

8. The retractable needle protector assembly as claimed in claim 7, wherein the opening made of a profile having a smaller diameter opening and a bigger diameter opening, wherein both the smaller diameter opening and bigger diameter opening adjoins in an area between the push interface and the locking member, wherein the smaller diameter opening part is adjacent the locking member and the bigger diameter opening part is adjacent the push interface.

9. The retractable needle protector assembly as claimed in claim 1, wherein the needle hub provided with a retaining element, a locking element and a blocking element in between proximal and distal end thereof.

10. The retractable needle protector assembly as claimed in claim 1, wherein the protective housing having an inner and outer wall, wherein the inner wall of the protective housing provided with a blocking member adjacent the proximal end of the protective housing.

11. The retractable needle protector assembly as claimed in claim 1, wherein the wing housing has a proximal opening and a distal opening to movably house the needle hub allowing the needle having the needle tip to pass through the distal opening thereof.

12. The retractable needle protector assembly as claimed in claim 2, wherein the wing housing has a proximal opening and a distal opening to movably house the needle hub allowing the needle having the needle tip to pass through the distal opening thereof.

13. The retractable needle protector assembly as claimed in claim 3, wherein the wing housing has a proximal opening and a distal opening to movably house the needle hub allowing the needle having the needle tip to pass through the distal opening thereof.

14. The retractable needle protector assembly as claimed in claim 2, wherein a spring is movably arranged within the wing housing over the needle hub in a distal region thereof.

15. The retractable needle protector assembly as claimed in claim 3, wherein a spring is movably arranged within the wing housing over the needle hub in a distal region thereof.

16. The retractable needle protector assembly as claimed in claim 4, wherein a spring is movably arranged within the wing housing over the needle hub in a distal region thereof.

17. The retractable needle protector assembly as claimed in claim 5, wherein a spring is movably arranged within the wing housing over the needle hub in a distal region thereof.
